(19) United States
(12) Patent Application Publication
(10) Pub. No.: US 2016/0062346 A1
(43) Pub. Date: Mar. 3, 2016

(54) System and Method for Customized Anatomical Implant Prosthetic Pieces

(71) Applicant: Sam Akmakjian, Long Grove, IL (US)

(72) Inventor: Sam Akmakjian, Long Grove, IL (US)

(21) Appl. No.: 14/835,116

(22) Filed: Aug. 25, 2015

Related U.S. Application Data
(60) Provisional application No. 62/041,998, filed on Aug. 26, 2014.

(51) Int. Cl. G05B 19/4099; A61C 8/00; A61C 13/00; A61B 6/14; A61B 6/03; A61C 5/10; A61C 13/08

(52) U.S. Cl. CPC G05B 19/4099; A61C 5/10; A61C 8/0037; A61C 13/08; A61B 6/14; A61B 6/032; A61C 13/0004; A61C 13/0007; A61C 2008/0046; G05B 2219/35134; G05B 2219/49007

(57) ABSTRACT

A system and method for creating customized anatomical implant prosthetic pieces that includes an x-ray computed tomography, a computer aided design component, and a computer aided manufacturing component. The method includes the steps of taking a tomographic image of an individual, generating a three-dimensional virtual anatomical model using the tomographic image, designing a three-dimensional virtual anatomical template using the three-dimensional virtual anatomical model, generating final information using the three-dimensional anatomical template, and generating a customized, patient-specific prosthetic piece from a solid block of material using the final information. The system and method of the present invention is fully performed and the prosthetic piece implanted into the patient within one office visit.

This application claims priority to provisional patent application Ser. No. 62/041,998, filed Aug. 26, 2014, to the extent allowed by law.

### BACKGROUND OF THE INVENTION

1. Field of the Invention

The present invention relates to a novel procedure in designing, fabricating, and placing customized anatomical titanium/titanium alloy implant prosthetic pieces utilizing an interface between a three-dimensional virtual anatomical model (3D VAM) fabricated by current X-Ray Computed Tomography (x-ray CT) technology and a computer aided design/computer aided manufacturing (CAD/CAM) milling system.

2. Description of the Prior Art

Implant prosthetics have become an increasingly popular part of prosthetic treatment, as they have been demonstrated to provide good anchorage to bone, providing a fixed platform on which an additional abutment can be placed to seat yet another prosthetic device. Current implants and abutments come in pre-determined sizes, and fail to conform to a patient's specific biological demands. None of these implants or abutments is shaped to resemble natural anatomy. Numerous shortcomings result from these mass-produced titanium fixtures, including poor integration, failure of the implant/abutment interface, poor tissue emergence, periodontal failures, etc. Such drawbacks create a demand for an improved technique that allows for superior esthetic outcomes.

Current computer aided design and computer aided manufacturing (CAD/CAM) technology allows for the customization of digital data. Once the CAD system receives information, it can digitally fabricate/alter that information. Modified information is then sent to a CAM portion, specifically a milling station, which will then reduce a solid block of substance to mold those exact parameters. Recent technologies in various development stages have tried to address creation of anatomic implants, either through additive technology, or a hybrid anatomical titanium implant fused to zirconium abutment. These technologies are dependent on outside laboratory processes that can take weeks to fabricate. Their cost is also much greater.

3D imaging currently integrated with milling devices is non-radiographic. Traditional 3D imaging is non-radiographic and only used for prosthetic components that are not biologically integrated, but rather are the prosthetic components. The system and method of the present invention integrates 3D x-ray technology with a milling unit to create surgical components that are placed in bone and creates surgically placed, biologically integrated, custom anatomical replica implants.

The system and method of the present invention may be used in other areas of medicine, including, but not limited to, the custom fabrication of articulating joints (hips, shoulders, knees, TMJ, etc.) and predominantly cartilaginous structures, such as ears and noses.

A primary object of the present invention is to provide a novel and improved technique that eliminates the need for assembling traditional, multi-piece prosthetic units.

It is another object of the present invention to provide an individual with a single, solid implant that will be more durable. The implant will reside within a natural tooth socket or within a manually created socket via osteotomy.

It is yet another object of the present invention to provide a custom implant that will nearly match the anatomy of each structure it will replace; such anatomical resemblance greatly improving the emergence profile of the final restoration, resulting in superior esthetics over traditional implants.

It is yet another object of the present invention to provide an implant where the conformity of the implant to the natural anatomical structures minimizes the bone recession that frequently occurs at the crestal-implant junction, providing such maintenance of bone integrity that will further enhance the retention of the custom implant by the bones of the maxilla/mandible of the patient.

It is yet another object of the present invention to provide better fitting implants with natural emergence profiles and a solid single piece implant and abutment with predetermined reduction of the coronal portion to allow for immediate crown placement.

It is yet another object of the present invention to provide prompt design, fabrication, and placement of the custom implant. The method of the present invention is the first in designing, fabricating, and placing a custom designed, bio-compatible root-based anatomical titanium implant during the course of a single visit to the dental office.

It is yet another object of the present invention to provide long-term cost reduction and time expediency. A patient will not have to wait for an off-site dental lab to manufacture and ship similarly designed custom implants. A patient can walk into an office and have the user design, fabricate, and place a custom implant prosthetic piece, with an attached prosthetic crown, all in a single visit to the office.

### SUMMARY OF THE INVENTION

The system and method for customized anatomical implant prosthetic pieces includes a system that comprises an x-ray computed tomography component, a computer aided design component, and a computer aided manufacturing component, such as a milling machine. The method of an illustrated embodiment of the present invention comprises the steps of taking at least one tomographic image including digital information of an individual using the x-ray computed tomography, sending at least one of the at least one tomographic image and digital information to a computer aided design component, generating a three-dimensional virtual anatomical model using at least one of the at least one tomographic image and digital information, modifying the three-dimensional virtual anatomical model into a three-dimensional virtual anatomical template using a set of criteria, generating final information using the modified three-dimensional virtual anatomical template that includes instructions for generating the prosthetic piece, sending the final information to the computer aided manufacturing component, and generating the prosthetic piece from a solid block of material using the final information.

In an alternate embodiment of the present invention, the method comprises the steps of taking at least one tomographic image including digital information of at least one of a first tooth and a bone socket of an individual using an x-ray computed tomography, sending at least one of the at least one tomographic image and the digital information to a computer aided design component, accessing a plurality of general, non-patient-specific anatomical models within the computer aided design component, selecting a general, non-patient-specific anatomical model of a second tooth from the plurality of general, non-patient-specific anatomical models within the computer aided design component, modifying a root portion of the general, non-patient-specific anatomical model to conform to the exact dimensions of one of the tooth and the bone socket of the individual as shown in the at least one tomographic image, modifying a crown portion of the general, non-patient-specific anatomical model to conform to the appropriate occlusal and interproximal clearance as shown in the at least one tomographic image, generating final information using the modified general, non-patient-specific anatomical model that includes instructions for generating the prosthetic piece, sending the final information to a computer aided manufacturing component, and generating the prosthetic piece from a solid block of material using the final information.

### BRIEF DESCRIPTION OF THE DRAWINGS

The system and method of the present invention is further described with reference to the accompanying drawings in which:

FIG. 1 is a front view of an individual's mouth, showing a fractured central incisor that is to be extracted.

FIG. 2 is a front view of a representative of a three-dimensional (3D) x-ray of the fractured central incisor.

FIG. 3A illustrates obtaining necessary measurements directly from the 3D x-ray.

FIG. 3B is a front plan view of a CAD milling machine.

FIG. 4A illustrates incorporation of the measurements in an initial design of a virtual anatomical model using computer aided design (CAD) software.

FIG. 4B illustrates modification of the initial design to take on a proper, but reduced, shape of a clinical crown that provides enough clearance for a porcelain cap.

FIG. 4C illustrates reduction of a solid block of titanium/titanium alloy to the specific measurements using computer aided machine (CAM) milling.

FIG. 4D illustrates a sand-blasted and polished custom implant.

FIG. 5 is a front view of the patient's mouth after extraction of the fractured central incisor.

FIG. 6A is a front view of the patient's mouth and the immediate placement of a custom implant following extraction.

FIG. 6B is a front view of the patient's mouth after a dental crown is fixed to the custom implant, completing restoration.

FIG. 7A is a side cross-sectional view of a tooth with a fractured crown.

FIG. 7B is a side cross-sectional view of a root of the tooth with the fractured crown.

FIG. 7C is a side cross-sectional view of the tooth with an anatomical crown.

FIG. 8 is a front view of the patient's mouth, showing an individual that is completely missing a maxillary central incisor.

FIG. 9 is a front view of the patient's mouth after an osteotomy has been performed.

FIG. 10 is a front view of a representative of a 3D x-ray of the osteotomy, where the dimensions of the socket are measured.

FIG. 11A illustrates the initial design of a custom implant to conform to the dimensions of the osteotomy using CAD software.

FIG. 11B illustrates modification of the initial design to take on the proper shape of the clinical crown, but reduced to provide enough clearance for a porcelain cap.

### DETAILED DESCRIPTION OF THE ILLUSTRATED EMBODIMENTS

The system and method of an illustrated embodiment of the present invention relates to a new technique for fabricating custom built implants that are designed to replicate any anatomical structure that may be replaced immediately following extraction. Requiring only one office visit to receive a custom designed titanium implant requires means to both fabricate and manufacture the anatomical implant prosthetic piece chairside. The system of the illustrated embodiment comprises an x-ray computed tomography, a computer aided design software component, and a computer aided manufacturing component. The computer aided design (CAD) software component and the computer aided manufacturing (CAM) component are combined into one CAD/CAM milling station that includes a CAM milling machine. The CAD/CAM milling station is designed to manipulate and transform an individual's digital information to recreate and design any anatomical structure that needs to be replaced, such as an individual's teeth, nose, ears, articulating joints, and other body parts.

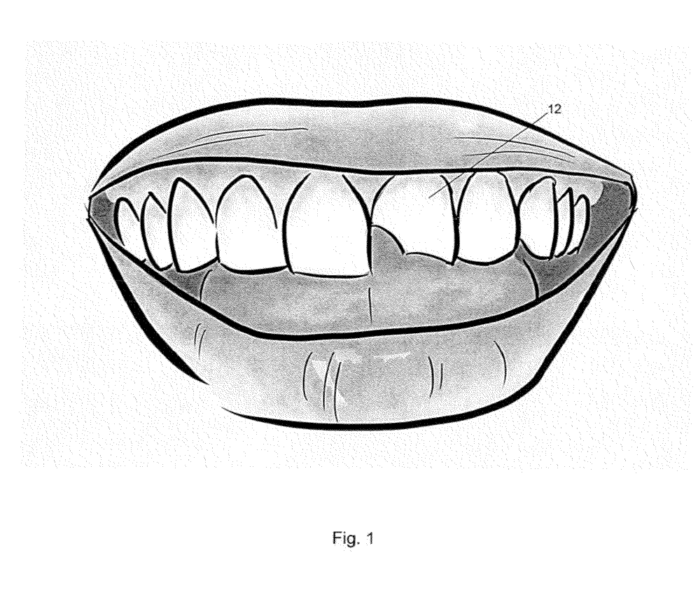

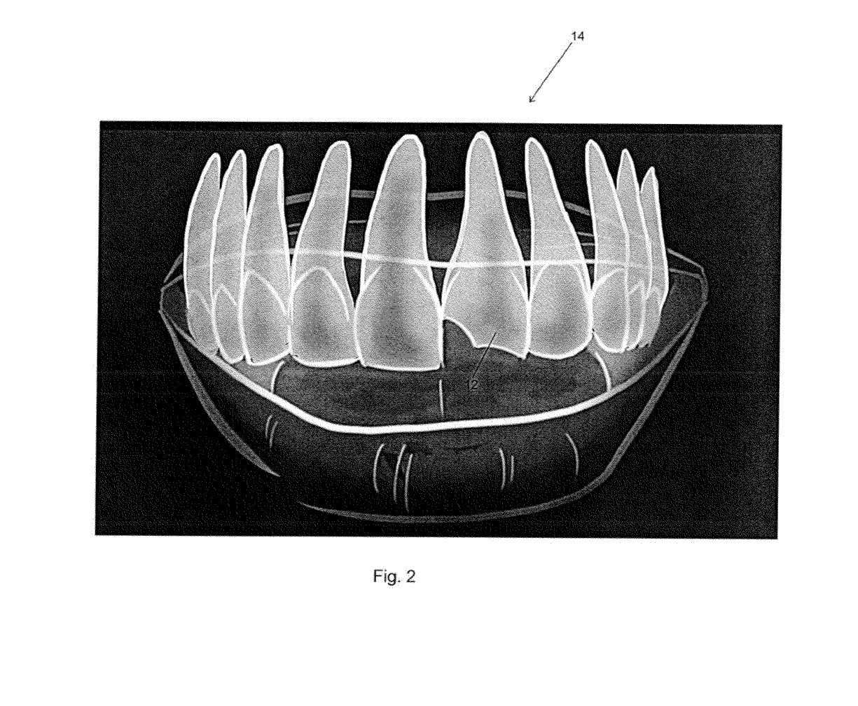

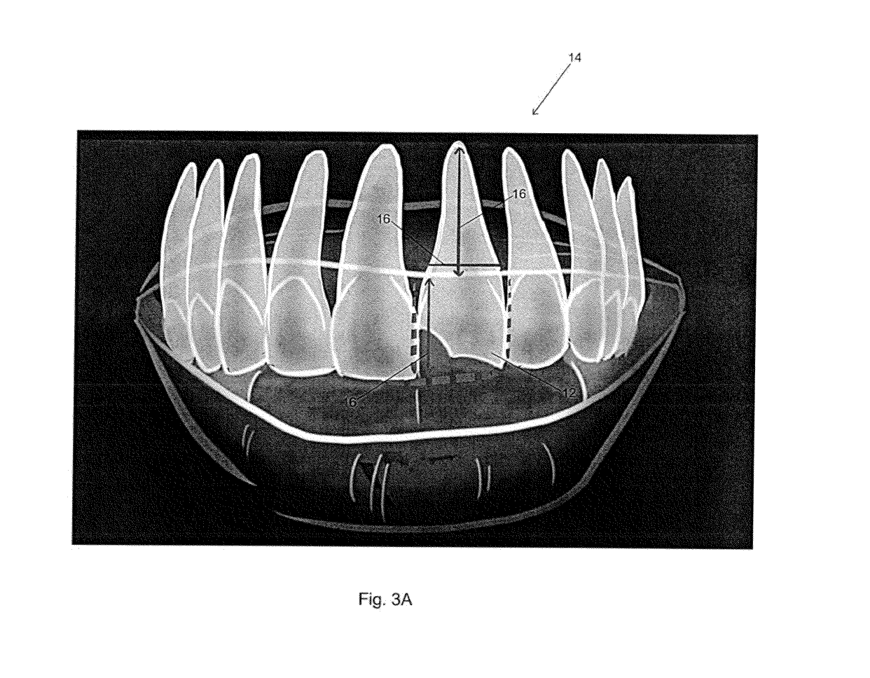

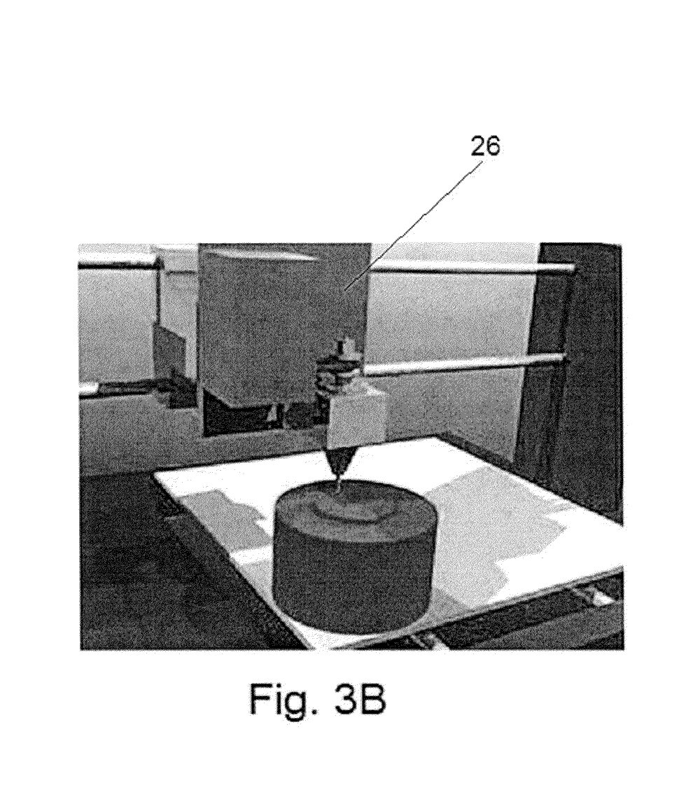

The method of the present invention is applied in the same way to any tooth, regardless of the anatomical form. The patient's exact tooth serves as a template for the fabrication of a single titanium/titanium alloy prosthetic piece that is custom-built according to an individual's specific biological demands. In a first illustrated embodiment, an individual's maxillary central incisor 12 is fractured, as shown in FIG. 1. At least one three-dimensional tomographic image (3D x-ray CT) 14 (FIG. 2), using the x-ray computed tomography component, is taken of an individual's mouth prior to extraction of the tooth 12. Any and all necessary original measurements 16 to reconstruct the affected tooth 12 are recorded as digital information directly from the 3D x-ray CT 14, as shown in FIG. 3A.

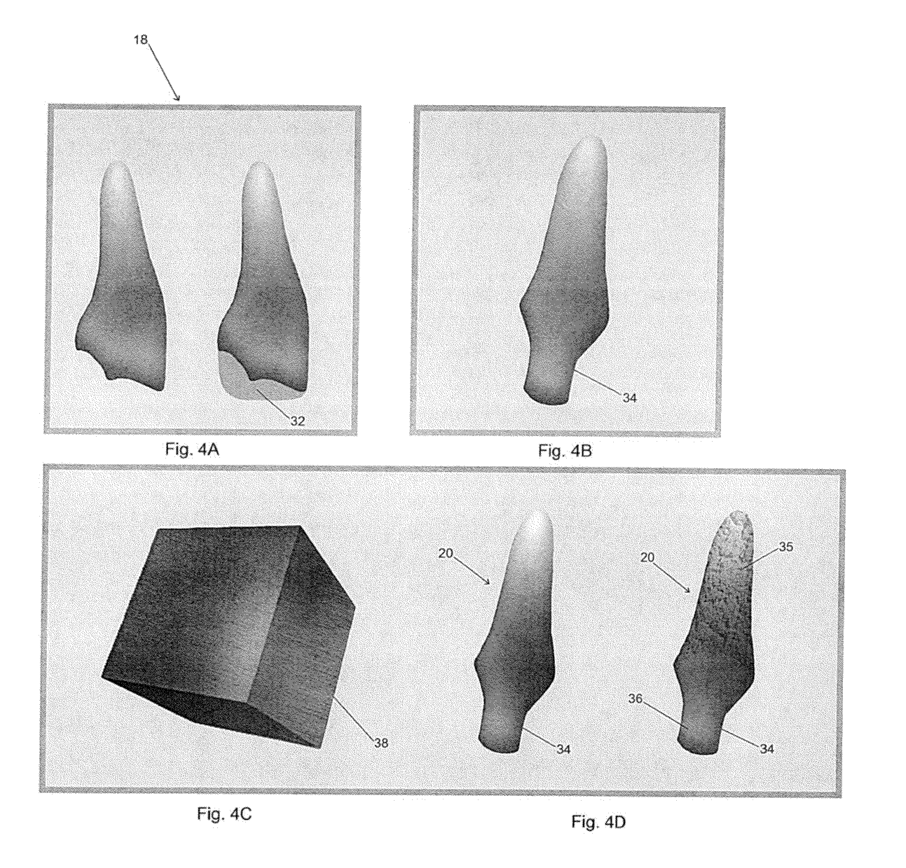

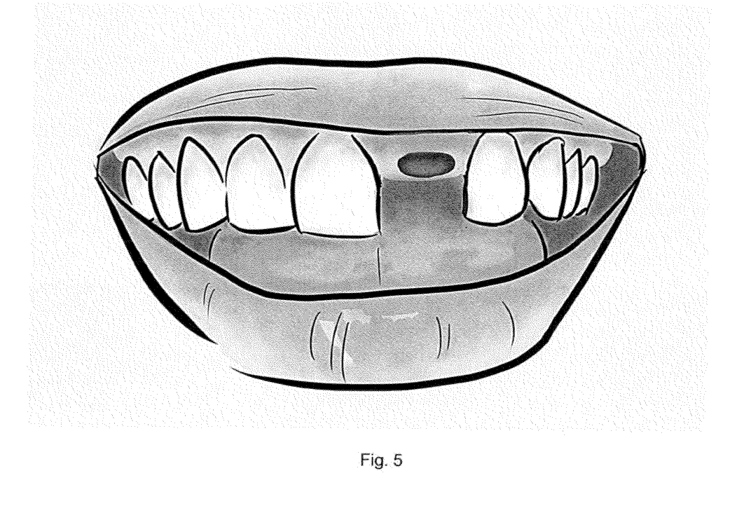

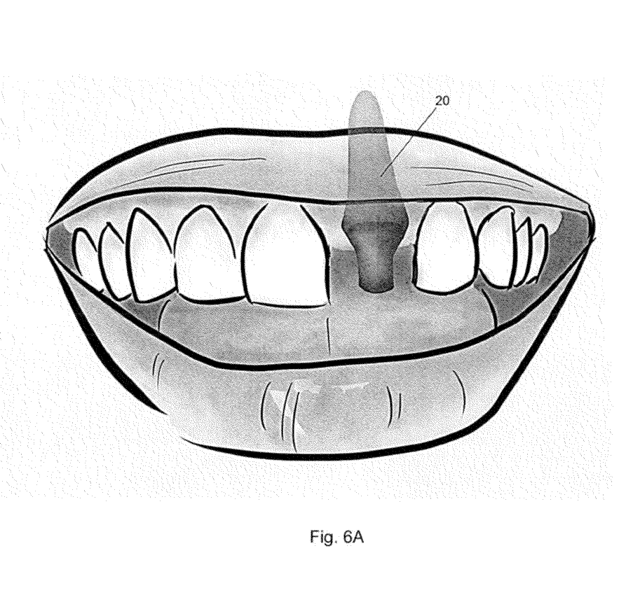

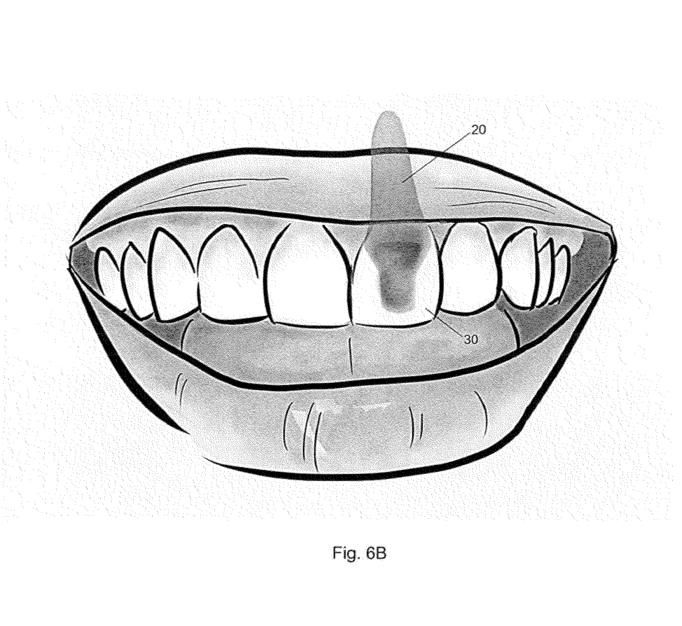

The digital information is subsequently sent to a CAD/CAM milling station 26, shown in FIG. 3B, where a three-dimensional virtual anatomical model (3D VAM) 18 is initially generated, as shown in FIG. 4A, using the 3D x-ray CT 14, the digital information, and the original measurements 16. The 3D VAM 18 is initially designed to conform to the anatomically-specific dimensions of the patient's tooth 12. The CAD software component of the CAD/CAM milling station 26 designs the accurate and precise 3D VAM 18 to take on the proper shape of the clinical crown 32 and subsequently adjusts the 3D VAM 18 so that adequate clearance is established for the placement of a second prosthetic piece, such as a dental crown 30, shown in FIG. 6B. Porcelain, or any variation of, generally requires a circumferential 1.5 mm shoulder and 2.0 mm incisal/occlusal reduction 34 for adequate clearance, as shown in FIGS. 4B and 4D. These measurements can be programmed as pre-set options within the CAD software component and can be further modified according to the user's desires.

The user can further modify the 3D VAM 18 to account for specific anatomical considerations that are found to optimize the placement and the osteo-integration of the custom anatomical implant. For example, dilacerated root shapes can be digitally modified prior to milling, divergent palatal roots can be eliminated prior to milling, and necessary clearance for the final prosthetic crown can be altered prior to milling. Modifying the 3D VAM 18 for specific anatomical considerations is of particular importance when replacing body parts that have unique emergence patterns from the body.

Once all modifications have been made to the 3D VAM 18, the final design of the 3D VAM 18 containing the accurate and precise instructions for building a biocompatible prosthetic piece 20, custom-designed to replicate any part of the natural anatomy, is subsequently sent to the CAD/CAM milling station 26. Titanium/titanium alloy blocks 38 are stocked in the user's office in various sizes that fit within the chamber of the CAD/CAM milling station 26. The block 38 is selected based on the size of the final design. The CAD/CAM milling station 26 reduces and carves the solid titanium/titanium alloy block 38, shown in FIG. 4C, to the exact final design parameters of the 3D VAM 18, producing a single prosthetic piece 20 that takes the near-exact anatomical shape of the patient's tooth 12, shown in FIG. 4D.

With current technology, the surface of the custom-built prosthetic piece 20 that will be in contact with the patient's bone can be modified to help enhance the patient's retention of the prosthetic piece. Such modifications include, but are not limited to, blasting the prosthetic piece (i.e., sand blasting using a large grit aluminum oxide), etching the prosthetic piece (i.e., using hydrochloric/sulfuric acid), coating the prosthetic piece with additive surface sprays (i.e., titanium plasma-sprayed surfaces, hydroxyapatite-coated surfaces, and platelet derived growth factors (PDGF)), and adding surface oxide layers to the prosthetic piece. In the present embodiment, the surface of the prosthetic piece 20 that will be in contact with the patient's bone is sand-blasted 35 to produce a rough surface on the root form and the remaining portion of the prosthetic piece 20 is polished, as shown in FIG. 4D.

The prosthetic piece 20 is then adequately sterilized prior to the placement of the prosthetic piece 20 in the patient's body. The prosthetic piece can be sterilized using steam autoclaving, ethylene oxide, ultraviolet (UV) light, laser exposure, and other adequate sterilization methods. The single, solid-metal prosthetic piece 20 produced by the CAD/CAM milling station 26 can then be used as a press-fit implant, placed immediately after extraction. Once the prosthetic piece 20 is ready for insertion, the fractured incisor 12 is extracted from the patient's mouth, as shown in FIG. 5, and the prosthetic piece 20 is placed in the patient's mouth, as shown in FIG. 6A. The custom prosthetic piece 20 can simultaneously serve as both an implant and an abutment for a dental crown 30 when placed in the patient's mouth, as shown in FIG. 6B.

The dental crown 30, designed and fabricated using the same CAD/CAM milling station 26 in the user's office, is then seated on top of the custom prosthetic piece 20 to complete the restoration, as shown in FIG. 6B. The user will exchange the titanium/titanium alloy block 38 with a porcelain block 40 inside the CAD/CAM milling station 26, or insert a porcelain block 40 in a second CAD/CAM milling station 26 that will fabricate the dental crown 30 simultaneously to the fabrication of the titanium/titanium alloy prosthetic piece 20. The method for creating this customized anatomical implant prosthetic piece is fully implemented in the user's office during a single office visit.

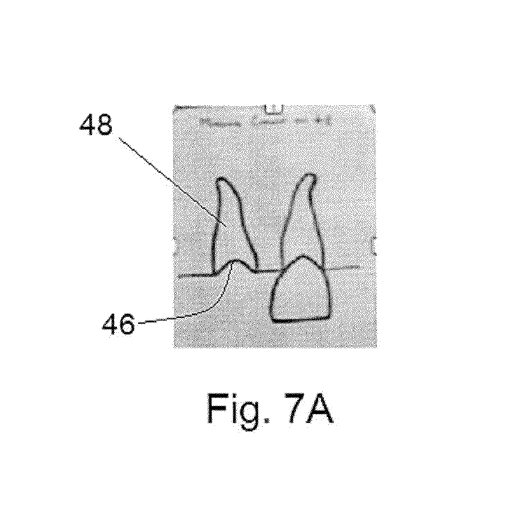

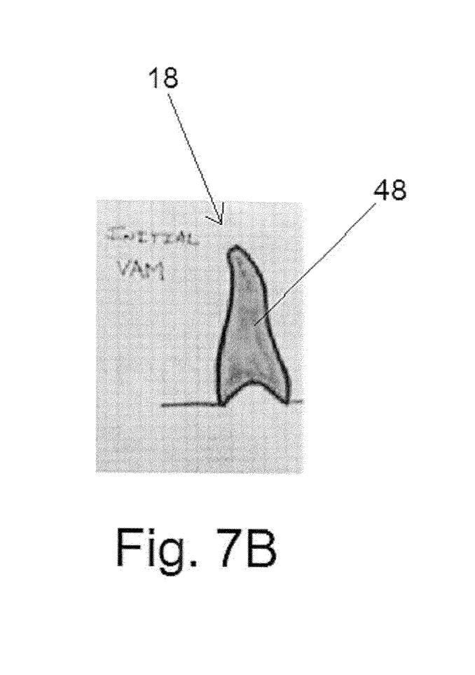

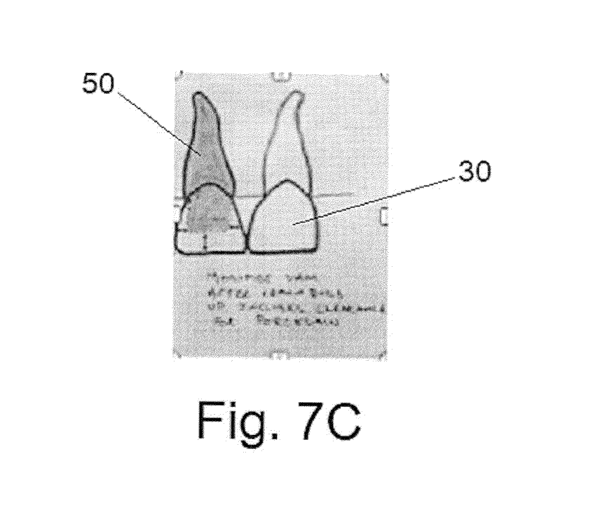

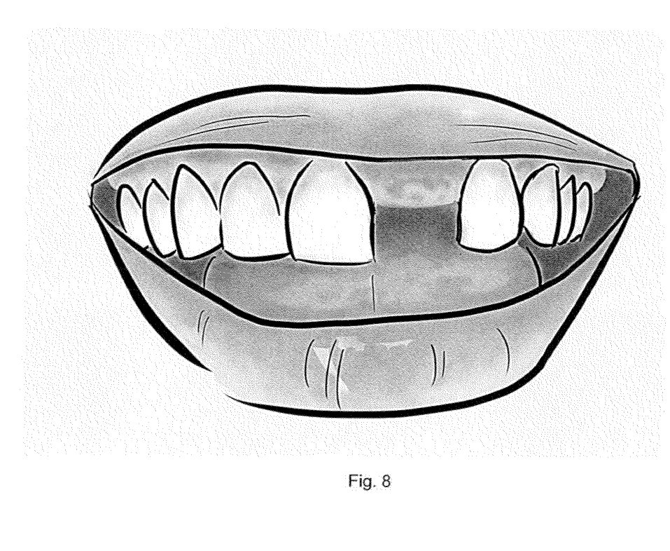

In a second illustrated embodiment of the method of the present invention, an individual presents with a fractured crown 46, leaving only root tips 48 intact, shown in FIGS. 7A-7C. In cases where an individual's natural clinical crown is unavailable to serve as part of the 3D VAM 18, the user will take a 3D x-ray CT 14 of the root tip 48 and use the 3D x-ray CT 14 as a model for preparing the prosthetic piece 20. The user will use the existing root structure as a template and will subsequently modify the model so that the prosthetic piece 20 takes on the form of the crown as it emerges out of the patient's bone. The initial 3D VAM 18, shown in FIG. 7B, will take on the shape of the patient's existing root form. Using the CAD software component, the user can then modify the 3D VAM 18 to add to the existing root form.

Within the CAD software component, the user will also have access to general anatomical models of all teeth. The user will then be able to select a general, non-patient-specific anatomical model 50 (FIG. 7C) and modify it in two steps. The first step is to modify the root portion of the model 50 so that it conforms to the exact dimensions of an individual's bone socket, as dictated by the findings of an individual-specific 3D x-ray CT 14, as shown in FIG. 7C. The second step is to modify the crown portion of the model 50 so that it displays appropriate occlusal and interproximal clearance, as dictated by the findings of the patient's 3D x-ray CT 14, for a prosthetic dental crown 30, also shown in FIG. 7C. The modifications will take on the shape of a matching anatomical crown being reduced occlusally and interproximally to seat a dental crown 30 that will also be fabricated and milled during the same office visit. This method allows the user to take any general model and make it patient specific.

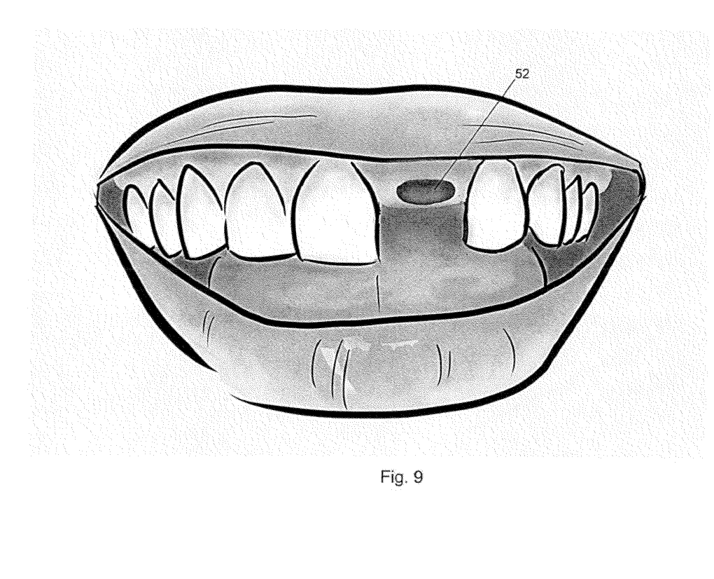

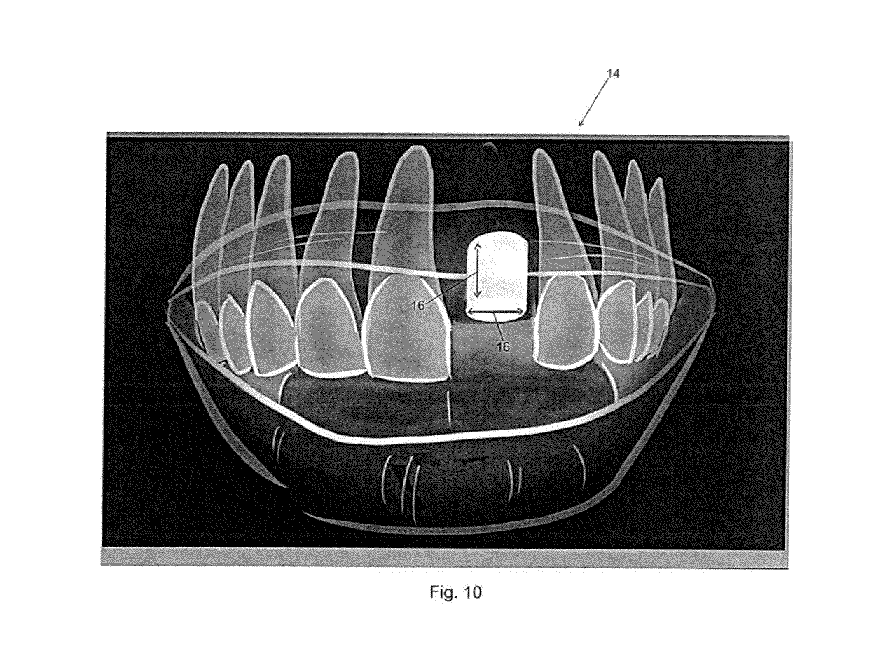

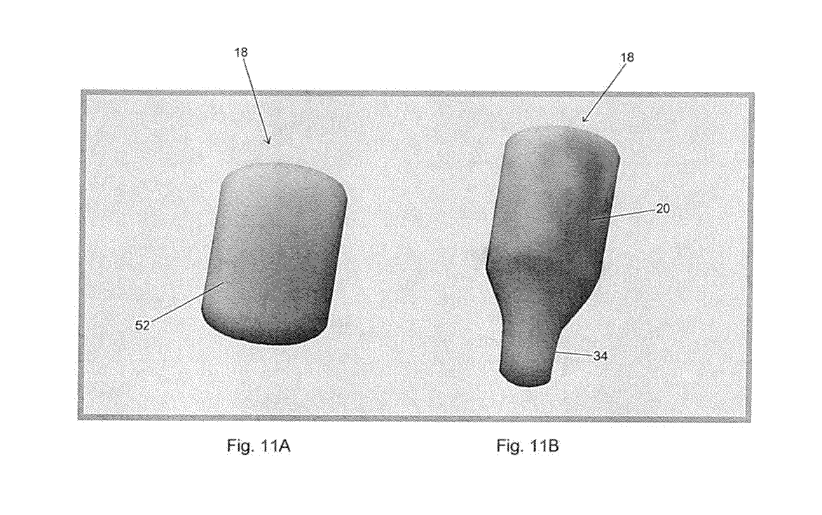

In a third embodiment of the method of the present invention, an individual presents with a missing tooth, such as a missing maxillary central incisor, as shown in FIG. 8. The dentist must perform an osteotomy on the patient and manually create a socket 52 for a prosthetic piece 20, as shown in FIG. 9. Once the osteotomy is performed, a 3D x-ray CT 14, as shown in FIG. 10, of the patient's mouth is taken in order to obtain the original measurements 16 of the prepared socket 52. The data obtained from the original measurements 16 is then used in the design of the 3D VAM 18 using the CAD software component. The 3D VAM 18 initially takes on the dimensions of the prepared socket 52, as shown in FIG. 11A, and is then modified so that the prosthetic piece 20 continues to take on the proper shape of the clinical crown 32, but reduced to provide enough clearance for the dental crown 30, as shown in FIG. 11B.

Modifications can be made to reshape the patient's existing bone, allowing for enhanced interaction between fabrication of the prosthetic piece and the placement of that prosthetic piece to allow for maximum esthetic control over unique emergence patterns. The 3D VAM 18 can be modified by the user to compensate for unusual anatomy and to alter emergence profiles. Additionally, single rooted implants can include the option to mill the implant with threads in the design process. The user can change the actual implant prosthesis manually through changes in the 3D VAM 18 prior to fabrication. Thus, the shape of the implant can be modified to conform to the user's preferences to idealize the final prosthesis. Single rooted implants can be threaded with parameters designed by the user to control thread width, thread depth, and pitch. Alternatively, the single rooted implant can be designed to be a press fit implant without threads.

The present invention is not limited to using the original measurements or the digital information captured from a 3D x-ray CT. Any non-vital structure that has been extracted intact from the patient's body can serve as an anatomical template, where simple CAD/CAM technology is coupled to existing oral scanners and digital imaging that take successive photographs and videos to create a three-dimensional virtual anatomical model. The method of the present invention allows a user to produce a custom anatomical implant prosthetic piece that exactly replicates an individual's unique anatomy into a single component: an implant and abutment fixture. The method is completed in the user's office and the prosthetic piece can be milled in minutes from the three-dimensional virtual anatomical model, without having to use an outside laboratory. The cost of creating these custom anatomical implant prosthetic pieces is a fraction of the cost of current mass-produced implants and prosthetic pieces, especially when the prosthetic portion or abutment component is added.

The foregoing description of illustrated embodiments of the invention has been presented for purposes of illustration and description, and is not intended to be exhaustive or to limit the invention to the precise form disclosed. The description was selected to best explain the principles of the invention and practical application of these principles to enable others skilled in the art to best utilize the invention in various embodiments and various modifications as are suited to the particular use contemplated. It is intended that the scope of the invention not be limited by the specification, but be defined by the claims set forth below.

What is claimed is:

1. A method for creating a customized anatomical prosthetic piece of a body part of an individual, the method comprising the steps of:
a. taking at least one tomographic image of the individual using an x-ray computed tomography, the at least one tomographic image including digital information;
b. sending at least one of the at least one tomographic image and the digital information to a computer aided design component;
c. generating a three-dimensional virtual anatomical model using at least one of the at least one tomographic image and the digital information;
d. modifying the three-dimensional virtual anatomical model into a three-dimensional virtual anatomical template based on a set of criteria;
e. generating final information using the modified three-dimensional virtual anatomical template, said final information comprising instructions for generating the prosthetic piece;
f. sending the final information to a computer aided manufacturing component; and
g. generating the prosthetic piece from a solid block of material using the final information.

2. The method of claim 1, wherein the three-dimensional virtual anatomical model conforms to the anatomically-specific dimensions of the body part of the individual.

3. The method of claim 1, wherein the set of criteria includes a plurality of measurements for reduction of the three-dimensional virtual anatomical model, the reduction adapted to provide adequate clearance for the placement of a prosthetic structure.

4. The method of claim 3, wherein the adequate clearance includes at least one of at least 1.5 mm circumferential shoulder reduction and at least 2.0 mm incisal/occlusal reduction.

5. The method of claim 1, wherein the set of criteria is user-defined.

6. The method of claim 1, further comprising the step of modifying the prosthetic piece that will be in contact with a bone of the individual to produce a textured surface by one of etching using at least one of hydrochloric acid and sulfuric acid, coating with additive surface sprays, and adding surface oxide layers.

7. The method of claim 1, further comprising the step of sand blasting the surface of the prosthetic piece that will be in contact with a bone of the individual.

8. The method of claim 1, further comprising the step of polishing the surface of the prosthetic piece that will not be in contact with a bone of the individual.

9. The method of claim 1, further comprising the step of sterilizing the prosthetic piece.

10. The method of claim 1, further comprising the step of sterilizing the prosthetic piece using at least one of steam autoclaving, ethylene oxide, ultraviolet light, and laser exposure.

11. The method of claim 1, wherein the final information is modified by adjusting the three-dimensional anatomical template to allow adequate clearance for the placement of a prosthetic structure

12. The method of claim 9, wherein the adequate clearance includes at least a circumferential 1.5 mm shoulder reduction.

13. The method of claim 9, wherein the adequate clearance includes at least a circumferential 2.0 mm incisal/occlusal reduction.

14. The method of claim 9, wherein the prosthetic structure is a dental crown.

15. The method of claim 1, wherein the computer aided manufacturing component is a milling machine.

16. The method of claim 1, wherein the prosthetic piece is one of a dental implant and a dental crown.

17. The method of claim 1, wherein the solid block of material is one of titanium, titanium alloy, and porcelain.

18. The method of claim 1, wherein the body part is a tooth.

19. A method for creating a customized anatomical prosthetic piece, the method comprising the steps of:
a. taking at least one tomographic image of at least one of a first tooth and a bone socket of an individual using an x-ray computed tomography, the at least one tomographic image including digital information;
b. sending at least one of the at least one tomographic image and the digital information to a computer aided design component;
c. accessing a plurality of general, non-patient-specific anatomical models within the computer aided design component;
d. selecting a general, non-patient-specific anatomical model of a second tooth from the plurality of general, non-patient-specific anatomical models within the computer aided design component;
e. modifying a root portion of the general, non-patient-specific anatomical model to conform to the exact dimensions of one of the tooth and the bone socket of the individual as shown in the at least one tomographic image;
f. modifying a crown portion of the general, non-patient-specific anatomical model to conform to the appropriate occlusal and interproximal clearance as shown in the at least one tomographic image;
g. generating final information using the modified general, non-patient-specific anatomical model, said final information comprising instructions for generating the prosthetic piece;
h. sending the final information to a computer aided manufacturing component; and
i. generating the prosthetic piece from a solid block of material using the final information.

20. The method of claim 19, wherein the prosthetic piece is one of a dental implant and a dental crown.

21. The method of claim 20, wherein the dental implant takes on the form of a dental crown on a first end.

22. The method of claim 19, wherein the solid block of material is one of titanium, titanium alloy, and porcelain.

23. The method of claim 19, further comprising the step of modifying the prosthetic piece that will be in contact with a bone of the individual to produce a textured surface by one of etching using at least one of hydrochloric acid and sulfuric acid, coating with additive surface sprays, and adding surface oxide layers.

24. The method of claim 19, further comprising the step of sand blasting the surface of the prosthetic piece that will be in contact with a bone of the individual.

25. The method of claim 19, further comprising the step of polishing the surface of the prosthetic piece that will not be in contact with a bone of the individual.

26. The method of claim 19, further comprising the step of sterilizing the prosthetic piece.

27. The method of claim 26, further comprising the step of sterilizing the prosthetic piece utilizing at least one of steam autoclaving, ethylene oxide, ultraviolet light, and laser exposure.

28. A system for creating a customized anatomical prosthetic piece, comprising:
a. an x-ray computed tomography for taking a tomographic image of an individual;
b. a computer aided design component for generating a three-dimensional virtual anatomical model containing digital information, designing a three-dimensional virtual anatomical template using at least one of the three-dimensional anatomical model and digital information, and generating final information using the three-dimensional virtual anatomical template, said final information comprising instructions for generating the prosthetic piece; and
c. a computer aided manufacturing component for generating the prosthetic piece from a solid block of material using the final information.

29. The system of claim 22, wherein the computer aided manufacturing component is a milling machine.

30. The system of claim 22, wherein the solid block of material is one of titanium, titanium alloy, and porcelain.

31. The system of claim 22, wherein the computer aided design component modifies the final information by adjusting the anatomical template to allow adequate clearance for the placement of a prosthetic structure.
